(19) United States
(12) Patent Application Publication
(10) Pub. No.: US 2007/0135909 A1
(43) Pub. Date: Jun. 14, 2007

(54) Adhesion polymers to improve stent retention

(76) Inventor: Jessica Renee DesNoyer, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/299,130

(22) Filed: Dec. 8, 2005

(51) Int. Cl. A61F 2/06

(52) U.S. Cl. 623/1.46

(57) ABSTRACT

It is disclosed a stent having a coating that contains one or more adhesion polymers that cause the stent not to unintentionally dislodge from a catheter on which the stent is mounted.

### BACKGROUND OF THE INVENTION

1. Field of the Invention

This invention generally relates to the use of an adhesive to prevent unintentional dislodgement of the stent from a catheter balloon.

2. Description of the Background

Percutaneous coronary intervention (PCI) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to radially compress against the atherosclerotic plaque of the lesion to remodel the lumen wall. The balloon is then deflated to a smaller profile to allow the catheter to be withdrawn from the patient's vasculature.

A stent can be implanted in conjunction with the balloon therapy to uphold luminal patentcy, to reduce or prevent the partial or total occlusion of the vessel caused by the collapse of intimal flaps or torn linings, and to reduce the chance of development of restenosis and thrombosis. Stent therapy can also be used as a means of local drug delivery. For example, a stent can include depots or channels containing a drug. A stent can also include a polymeric coating containing a drug for local release of the drug.

A stent is collapsed and placed or mounted over a balloon of a catheter. Both bare metal stents and polymer coated stents can become dislodged from the balloon prior to deployment of the stent at the treatment site. Stents made from bioabsorbable polymers, with or without a coating, can similarly harbor dislodgement problems. The dislodgement of a stent becomes even more problematic if a polymer coating provides for less friction on the balloon surface than a bare metal stent or when the polymer is “slippery” in nature or becomes “slippery” when exposed to biological fluids. One method of preventing unintentional dislodgement is by providing mechanical folds or tabs on the balloon so as to physically hold the stent in place. Various stent crimping procedures have also been proposed by those skilled in the art to hold the stent in place, such as a two phase crimping process using various temperatures. Changing the shape of balloon designs or providing for a rigorous stent-balloon attachment process can be expensive, time consuming, and in certain cases can cause damage to a polymeric coating on the stent. Coating defect caused by such damage can lead to adverse biological responses.

The embodiments of the present invention provide for methods addressing these issues.

### SUMMARY OF THE INVENTION

Provided herein is a process for improving stent retention using an adhesion polymer that improves adhesion to the catheter balloon. In some embodiments, the process includes forming a layer that contains a substantial amount of the adhesion polymer as a topcoat over an underlying stent coating. In some embodiments, the adhesion polymer can be included in a coating layer on the stent, but needs to be in a substantial amount so as to perform its intended function of preventing unintentional dislodgement. The adhesion polymer topcoat or the coating including the adhesion polymer can be coated conformally or only on the luminal stent surface in contact with the catheter balloon. The underlying stent coating may include one or more layers of other materials which may or may not include the adhesion polymer. The coating construct thus formed has a sufficient retention such that the stent does not become dislodged from the catheter prior to stent deployment. In some embodiments, to achieve the same desired effect, the adhesion polymer can be applied as a coating over the catheter balloon rather than or in addition to the use of the adhesion polymer on the stent.

The stent can be a metallic, biodegradable or nondegradable stent. The stent can be intended for neurovasculature, carotid, coronary, pulmonary, aortic, renal, biliary, iliac, femoral, popliteal, or other peripheral vasculature. The stent can be used to treat, prevent or ameliorate a disorder such as atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, or tumor obstruction.

### BRIEF DESCRIPTION OF THE DRAWINGS

FIG. 1 shows the luminal surface of PEA-BZ construct post simulated use.

FIG. 2 shows the luminal surface of PEA-TEMPO construct post simulated use.

### DETAILED DESCRIPTION

Provided herein is a process for improving stent retention using an adhesion polymer that improves adhesion to a catheter balloon. Catheter balloons are very well known to those of ordinary skill in the art. In some or all embodiments, the adhesion polymer is applied in a substantial amount to at least the inner surface of the stent. In some embodiments, the adhesion polymer is blended, combined, mixed, conjugated or chemically bonded to a polymer included in a coating on the stent. The bonding can be to a polymer backbone or a pendant group. In some embodiments, surface attachment is preferable. In these embodiments, the adhesion polymer must be presented in a substantial amount so as to perform its intended function and/or be present mostly on the outer surface of the coating so that its intended function is achieved. In some embodiments, the process includes forming a layer that contains or includes a substantial amount of the adhesion polymer as a topcoat over an underlying stent coating. The underlying stent coating may include one or more layers of other materials which may or may not include the adhesion polymer. In some embodiments, a surface of the balloon can be coated with a substantial amount of a layer including the adhesion polymer. This balloon coating can be used in lieu of or in conjunction with the use of the adhesion polymer with the stent. The coating construct thus formed has a sufficient retention such that the stent does not become unintentionally dislodged from the catheter prior to stent deployment.

Stent deployment means the start of the expansion or inflation process of the balloon of the catheter as is understood by one of ordinary skill in the art. Accordingly, the stent does not unintentionally dislodge subsequent to mounting or crimping of the stent on the balloon as well as directing the stent on the catheter to the area in need of treatment.

Unintentional dislodgement means that the stent moves less than 3 mm, 2 mm, 1 mm, 0.5 mm, 0.25 mm, 0.2 mm, 0.15 mm, 0.1 mm, 0.05 mm, 0.025 mm, 0.01 mm, or 0.001 mm in linear direction (sliding back and/or forth on the balloon) and/or radial direction (rotating with respect to the balloon) subsequent to crimping or mounting the stent on the balloon and prior to the start of the inflation or expansion of the balloon. Preferably the movement should be limited to less than 1 mm. More preferably, the movement should be limited to less than 0.5 mm. Most preferably, the movement should be limited to less than 0.25 mm, 0.2 mm, or 0.15 mm.

As used herein, the term “a substantial amount” means about or above 20%, about or above 50%, about or above 60%, about or above 70%, about or above 75%, about or above 80%, about or above 85%, about or above 90%, about or above 95%, or about 100% of the total amount of the material forming the coating layer or topcoat layer. For example, if the adhesion material is used in a coating layer for a stent and not as a topcoat layer, the amount of adhesion material can be, for example, above 90% in the coating layer by mass.

In some embodiments, the term “adhesion polymer”, as used herein, refers to a polymer that (1) has a glass transition temperature between about 25° C. and about 45° C. and/or (2) has hydrogen-bonding groups that can interact with the hydrogen bonding groups in the balloon material. In addition, the adhesive interaction between a coating containing the polymer and the catheter balloon cannot be so great that the mechanical integrity of the coating is compromised.

T_{g} as used herein generally refers to the temperature at which the amorphous domains of a polymer change from a brittle vitreous state to a plastic state at atmospheric pressure. In other words, T_{g} corresponds to the temperature where the onset of segmental motion in the chains of the polymer occurs, and it is discernible in a heat-capacity-versus-temperature graph for a polymer. When an amorphous or semicrystalline polymer is heated, its coefficient of expansion and heat capacity both increase as the temperature rises, indicating increased molecular motion. As the temperature rises, the sample's actual molecular volume remains constant. Therefore, a higher coefficient of expansion points to a free volume increase of the system and increased freedom of movement for the molecules.

Polymers useful as the adhesion polymer described herein can be any biocompatible polymer having hydrogen bonding groups such as carboxyl, carboxylate, amine, amide, phosphoryl, phosphate, sulfonic, sulfate, OH, SH, or combinations thereof. Such biocompatible polymers can be made from monomers bearing carboxyl, amine, amide, phosphoryl, phosphate, sulfonic, sulfate, OH, or SH groups, which can be homo- or copolymers.

Some representative adhesion polymers include, but are not limited to, poly(ester amides) (PEA), polyanhydride, polyacids such as poly(acrylic acid), poly(methacrylic acid), poly(vinyl alcohol), and combinations thereof. In some embodiments of the invention, a provision is being provided that the adhesion polymer can specifically exclude any one the aforementioned polymers.

In some embodiments, the adhesion polymer is PEA. PEA encompasses a polymer having at least one ester grouping and at least one amide grouping in the backbone. One example is the PEA polymer made according to Scheme I. Other PEA polymers are described in U.S. Pat. No. 6,503,538 B1. An example of the PEA polymer includes diacid, diol, and amino acid subunits, e.g., co-poly-{[N,N′-sebacoyl-bis-(L-leucine)-1,6-hexylene diester]-[N,N′-sebacoyl-L-lysine benzyl ester]} (PEA-Bz) and co-poly{[N,N′-sebacoyl-bis-(L-leucine)-1,6-hexylene diester]-[N,N′-sebacoyl-L-lysine 4-amino-TEMPO amide]}(PEA-TEMPO) ), which have a T_{g} of approximately 23° C. and 33° C., respectively.

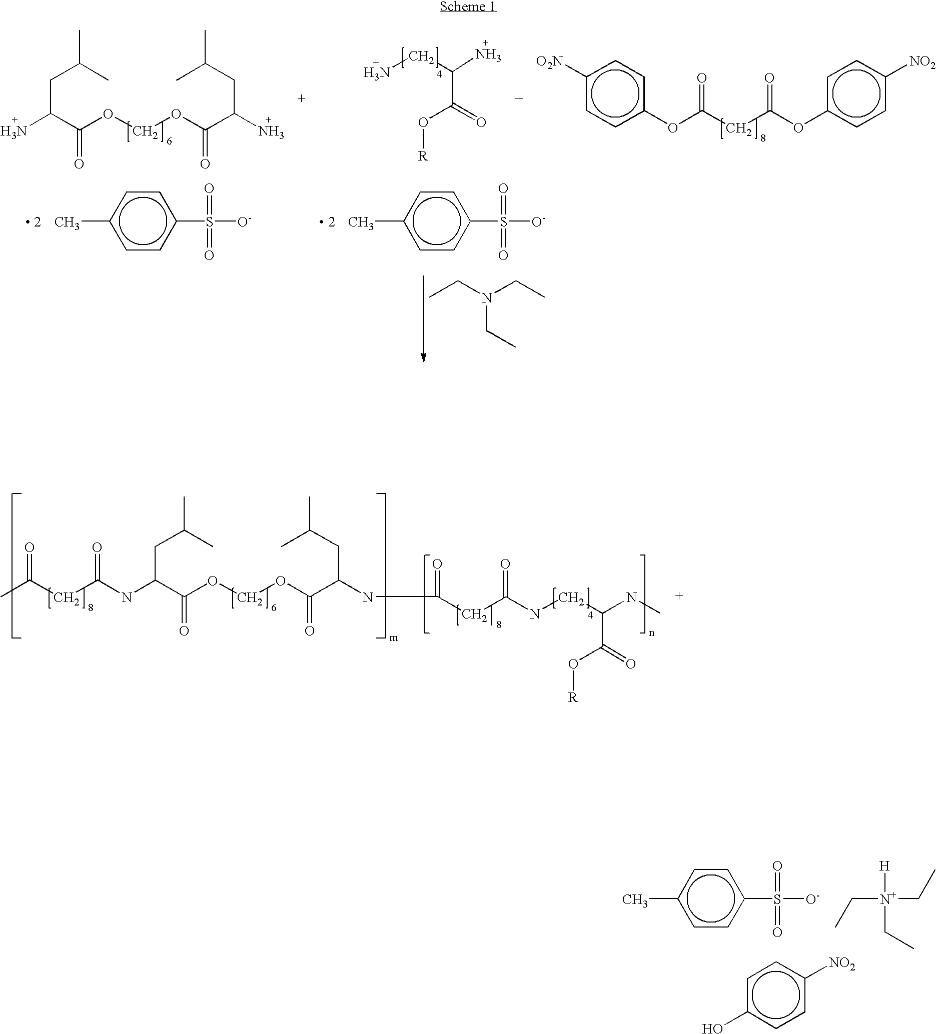

PEA polymers can be made by condensation polymerization utilizing, among others, diacids, diols, diamines, and amino acids. Some exemplary methods of making PEA are described in U.S. Pat. No. 6,503,538 B1.

The adhesion polymer can be used alone or with one or more other biocompatible polymers. When it is used in conjunction with other polymers, the adhesion polymer content should be sufficiently high such that a coating thus formed meets the adhesion requirements described above. Additionally, the adhesion polymer may need to be present on the outermost surface of the coating at a sufficient amount so as to perform its intended function. While a coating may contain polymers otherwise useful as adhesion polymers, the mere existence of such polymers in the coating is not necessarily sufficient to cause the coating to meet the aforementioned adhesion requirements. To serve as an adhesion polymer, a polymer otherwise useful as a adhesion polymer should be distributed within the coating to afford enough of such polymer molecules at the surface of the coating to cause the coating to meet the adhesion requirements. That is, without being bound by any particular theory, the coating meets the adhesion requirements primarily due to the presence of adhesion polymer molecules distributed at or near the surface of the coating. Alternatively, to serve as an adhesion polymer, a polymer otherwise useful as an adhesion polymer may be present at a concentration sufficient to modify the properties of the other coating components such that the coating meets the adhesion requirements.

The adhesion polymer can be used optionally with a biobeneficial material and/or a bioactive agent to coat a stent. In some other embodiments, the adhesion polymer can be used with one or more biocompatible polymers, which can be biodegradable, bioabsorbable, bioerodable, non-degradable, or non-bioabsorbable polymers. Biodegradable, bioabsorbable, and bioerodable are terms which are used interchangeably unless otherwise specifically indicated. As previously indicated, the adhesion polymer can be coated onto a stent as a topcoat, with or without other layers of coating. The topcoat can be a layer with or without a bioactive agent or a drug. The other layers can include a drug or bioactive agent. In one embodiment, the adhesion polymer can be used in both the topcoat and the drug matrix layer of the coating, with or without another biocompatible polymer.

The stent can be metallic or polymeric, either biodegradable or non-degradable. The stent can be intended for neurovasculature, carotid, coronary, pulmonary, aortic, renal, biliary, iliac, femoral, popliteal, or other peripheral vasculature. The stent can be used to treat or prevent a disorder such as atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, or tumor obstruction.

Some examples of the biocompatible polymer and/or biobeneficial materials that can be used with the adhesion polymer described herein include, but are not limited to, ethylene vinyl alcohol copolymer (commonly known by the generic name EVOH or by the trade name EVAL), poly(hydroxyvalerate), polycaprolactone, poly(lactide-co-glycolide), poly(hydroxybutyrate), poly(hydroxybutyrate-co-valerate), polydioxanone, poly(glycolic acid-co-trimethylene carbonate), polyphosphoester urethane, poly(amino acids), polycyanoacrylates, poly(trimethylene carbonate), poly(iminocarbonate), polyurethanes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as vinylidene fluoride based home or copolymer under the trade name Solef™ or Kynar™, for example, polyvinylidene fluoride (PVDF) or poly(vinylidene-co-hexafluoropropylene) (PVDF-co-HFP) and polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), epoxy resins, polyurethanes, rayon, rayon-triacetate, cellulose acetate, cellulose butyrate, cellulose acetate butyrate, cellophane, cellulose nitrate, cellulose propionate, cellulose ethers, and carboxymethyl cellulose, copolymers of these polymers with poly(ethylene glycol) (PEG), or combinations thereof. Again, the adhesion polymer can be in a layered construct or blended with any of the aforementioned polymers or other polymers described herein after. For example, the stent can include a polymer layer including PVDF-co-HFP and a drug as well as a layer including an adhesion polymer deposited thereon. In some embodiments, attachment to the surface of a coating made from such polymers may be preferable.

In some embodiments, the biocompatible polymer can be poly(ortho esters), poly(anhydrides), poly(D,L-lactic acid), poly (L-lactic acid), poly(glycolic acid), copolymers of poly(lactic) and glycolic acid, poly(L-lactide), poly(D,L-lactide), poly(glycolide), poly(D,L-lactide-co-glycolide), poly(L-lactide-co-glycolide), poly(phospho esters), poly(trimethylene carbonate), poly(oxaesters), poly(oxaamides), poly(ethylene carbonate), poly(propylene carbonate), poly(phosphoesters), poly(phosphazenes), poly(tyrosine derived carbonates), poly(tyrosine derived arylates), poly(tyrosine derived iminocarbonates), copolymers of these polymers with poly(ethylene glycol) (PEG), or combinations thereof.

In some other embodiments, the biocompatible polymer can exclude any one or more of the polymers provided above.

The biocompatible polymer can provide a controlled release of a bioactive agent and/or binding of the bioactive agent to a substrate, which can be the surface of a stent or a coating thereon. Controlled release and delivery of bioactive agent using a polymeric carrier has been extensively researched in the past several decades (see, for example, Mathiowitz, Ed., Encyclopedia of Controlled Drug Delivery, C.H.I.P.S., 1999). For example, PLA based drug delivery systems have provided controlled release of many therapeutic drugs with various degrees of success (see, for example, U.S. Pat. No. 5,581,387 to Labrie, et al.). The release rate of the bioactive agent can be controlled by, for example, selection of a particular type of biocompatible polymer, which can provide a desired release profile of the bioactive agent. The release profile of the bioactive agent can be further controlled by selecting the molecular weight of the biocompatible polymer and/or the ratio of the biocompatible polymer to the bioactive agent. One of ordinary skill in the art can readily select a carrier system using a biocompatible polymer to provide a controlled release of the bioactive agent.

A preferred biocompatible polymer is a polyester, such as one of PLA, PLGA, PGA, PHA, poly(3-hydroxybutyrate) (PHB), poly(3-hydroxybutyrate-co-3-hydroxyvalerate), poly((3-hydroxyvalerate), poly(3-hydroxyhexanoate), poly(4-hydroxybutyrate), poly(4-hydroxyvalerate), poly(4-hydroxyhexanoate), polycaprolactone (PCL), poly(ester amide), polyvinylidene halides or a combination thereof.

The bioactive agents can be any agent which is a therapeutic, prophylactic, or diagnostic agent. These agents can have anti-proliferative or anti-inflammmatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include 40-epi-(N1-tetrazolyl)-rapamycin (ABT-578), 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax ä (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of anti-inflammatory agents including steroidal and non-steroidal anti-inflammatory agents include tacrolimus, dexamethasone, clobetasol, combinations thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include pimecrolimus, imatinib mesylate, midostaurin, alpha-interferon, bioactive RGD, and genetically engineered epithelial cells. The foregoing substances can also be used in the form of prodrugs or co-drugs thereof. The foregoing substances are listed by way of example and are not meant to be limiting. Other active agents which are currently available or that may be developed in the future are equally applicable.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent required to inhibit the desired cellular activity of the vascular region can depend upon factors such as the particular circumstances of the patient; the nature of the trauma; the nature of the therapy desired; the time over which the ingredient administered resides at the vascular site; and if other active agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

As used herein, a stent may be any suitable medical substrate that can be implanted in a human or veterinary patient. Examples of such stents include, unless otherwise specifically stated, self-expandable stents, balloon-expandable stents, stent-grafts, and grafts (e.g., aortic grafts). The underlying structure of the stent can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention. In one embodiment, the stent can be a bioabsorbable polymer, without a drug coating, having the adhesive polymer on at least the inner surface of the stent. In some embodiments, the bioabsorbable polymer stent can include a polymer drug coating containing the adhesive polymer and/or coated with the adhesive polymer.

For coatings including one or more active agents, the agent will remain on the stent during delivery and expansion of the device, and be released at a desired rate and for a predetermined duration of time at the site of implantation. Preferably, the medical device is a balloon expandable stent. A stent having the above-described coating is useful for a variety of medical procedures, including, by way of example, treatment of obstructions caused by tumors in bile ducts, esophagus, trachea/bronchi and other biological passageways. A stent having the above-described coating is particularly useful for treating occluded regions of blood vessels caused by abnormal or inappropriate migration and proliferation of smooth muscle cells, thrombosis, and restenosis. Stents may be placed in a wide array of blood vessels, both arteries and veins. Representative examples of sites include the iliac, renal, and coronary arteries.

For implantation of a stent, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter, which allows a stent in its collapsed configuration to be inserted into the passageway. The delivery catheter is inserted either percutaneously or by surgery into the femoral artery, brachial artery, femoral vein, or brachial vein, and advanced into the appropriate blood vessel by steering the catheter through the vascular system under fluoroscopic guidance. A stent having the above-described coating may then be expanded at the desired area of treatment. A post-insertion angiogram may also be utilized to confirm appropriate positioning.

### EXAMPLES

The embodiments of the present invention will be illustrated by the following set forth examples. All parameters and data are not to be construed to unduly limit the scope of the embodiments of the invention.

### Example 1

12 mm small Vision stents (available from Guidant Corporation) were spray-coated with PEA-BZ in a 2-layer construct design as follows:

Layer 1: 616 μg of PEA-BZ/everolimus (drug:polymer=1:10);

Layer 2: 384 μg of PEA-BZ.

12 mm Small Vision stents were spay-coated with PEA-TEMPO in a 2-layer construct design as follows:

Layer 1: 392 μg of PEA-TEMPO/everolimus (drug:polymer=1:6);

Layer 2: 400 μg of PEA-TEMPO.

12 mm Small BMS Vision stents (available from Guidant Corporation) were used as controls.

All stents were E-beam sterilized (25 KGy, single pass, under argon) following the crimp and a temperature variable stent-catheter attachment processes.

The stent retention of these units was tested in the stent dislodgement test. Stent dislodgement is measured by attaching a stent to an Instron device and monitoring the load required to remove the stent from the catheter. The typical acceptance range for stent dislodgement is 0.8-1.2. The standard sample size is n=5/arm.

The average distal maximum loads required to remove each of the study groups from catheters are summarized in Table 1. The Vision control arm falls outside of the acceptance range because an additional stent retention process step, stent press, was not performed. Applying PEA-BZ and PEA-TEMPO coatings brings the stent retention into the acceptance range without performing this additional stent retention process step.

TABLE 1
Stent dislodgement test results

| | Avg Distal | | | | |
|---|---|---|---|---|---|
| Arm | Load | St Dev | Min | Max | Comments |
| BMS | 0.573 | 0.467 | 0.344 | 1.407 | — |
| Vision | | | | | |
| PEA-BZ | 1.059 | 0.203 | 0.810 | 1.252 | Stents could not be |
| | | | | | removed from catheter. |
| | | | | | Recorded pull force |
| | | | | | value at which distal |
| | | | | | stent struts were |
| | | | | | damaged. |
| PEA- | 0.836 | 0.202 | 0.5 | 0.987 | Pull force eventually |
| TEMPO | | | | | breaks stent retention |
| | | | | | and causes an abrupt |
| | | | | | move to distal end. |

PEA-BZ has a lower T_{g} than PEA-TEMPO and, therefore, is more adhesive than PEA-TEMPO, giving an even greater improvement in stent retention.

Example 2

12 mm Small Vision stents were spray-coated with PEA-BZ in a 2-layer construct design as follows: Layer 1: 616 μg PEA-BZ/everolimus (drug:polymer=1 :10); Layer 2: 384 μg PEA-BZ.

12 mm Small Vision stents were spray coated with PEA-TEMPO in a 2-layer construct design as follows: Layer 1: 392 μg PEA-TEMPO/everolimus (drug:polymer=1:6); Layer 2: 400 μg PEA-TEMPO.

Following the crimp a temperature variable stent-catheter attachment process, all units were E-beam sterilized (25 KGy, single pass, under argon).

The units were tested in the simulated use experiment to determine the effect of delivery and deployment on coating integrity. In this experiment, a stent is guided through a tortuous path and then deployed in a 3.0 mm×4 inch poly(vinyl alcohol) (PVA) lesion. The tortuous tubing and the PVA lesion contain deionized (DI) water (T=37° C.). After deployment and catheter retraction, DI water (T=37° C.) is pumped through the apparatus at 50 mL/min. for 1 hr. The units are subsequently analyzed by scanning electron microscopy (SEM).

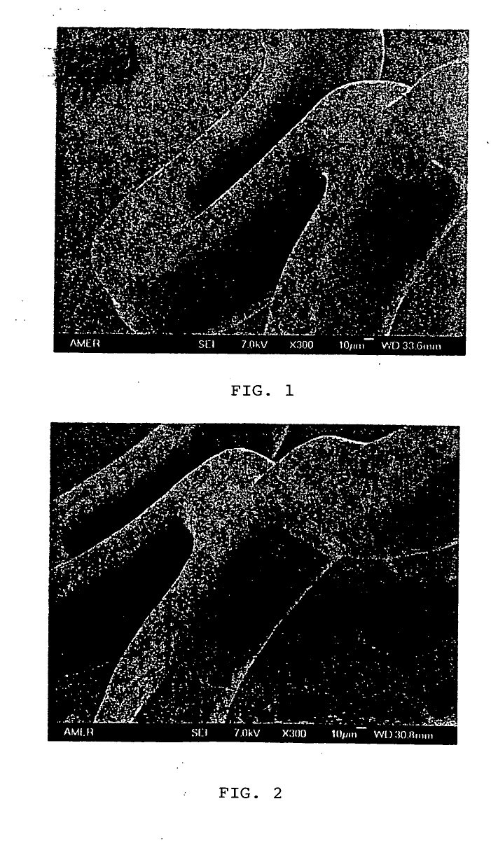

Though both polymers were shown to improve stent retention in example 1, neither interact with the catheter balloon to an extent that coating mechanical integrity is compromised (FIG. 1 and FIG. 2).

While particular embodiments of the present invention have been shown and described, it will be obvious to those skilled in the art that changes and modifications can be made without departing from this invention in its broader aspects. Therefore, the appended claims are to encompass within their scope all such changes and modifications that fall within the true spirit and scope of this invention.

What is claimed is:

1. A stent comprising a coating having an adhesion polymer in an amount effective to cause the stent not to unintentionally dislodge from a catheter balloon on which the stent is mounted.

2. The stent of claim 1, wherein unintentional dislodgement is defined as less than 1 mm in linear and/or radial movement by the stent on the balloon prior to the start of the inflation or expansion of the balloon.

3. The stent of claim 1, wherein unintentional dislodgment is defined as less than 0.5 mm in linear and/or radial movement by the stent on the balloon prior to the start of the inflation or expansion of the balloon.

4. The stent of claim 1, wherein the adhesion polymer has a glass transition temperature between about 25° C. and about 45° C.

5. The stent of claim 1, wherein the adhesion polymer is formed of monomers comprising a hydrogen bonding group.

6. The stent of claim 5, wherein the hydrogen bonding group is selected from the group consisting of carboxyl, carboxylate, amine, amide, phosphoryl, phosphate, sulfonic, sulfate, hydroxyl (OH), thiol (SH), or combinations thereof.

7. The stent of claim 1, wherein the adhesion polymer is selected from the group consisting of poly(ester amide), polyanhydride, polyacid, poly(acrylic acid), poly(methacrylic acid), poly(vinyl alcohol), and combinations thereof.

8. The stent of claim 1, wherein the adhesion polymer comprises PEA-BZ.

9. The stent of claim 1, wherein the adhesion polymer comprises PEA-TEMPO.

10. The stent of claim 1, wherein the adhesion polymer is included in a topcoat layer over a coating layer on the stent.

11. The stent of claim 1, wherein the adhesion polymer is limited to an inner surface of the stent and not an outer surface of the stent.

12. The stent of claim 1, wherein the stent is bioabsorbable and the adhesion polymer is disposed on the inner surface of the bioabsorbable stent.

13. The stent of claim 1, wherein the adhesion polymer is blended, combined, mixed, conjugated or chemically bonded to a polymer of a coating for the stent.

14. A stent-catheter medical assembly comprising a stent mounted on a balloon catheter, wherein (1) an inner side of the stent includes an adhesion polymer, (2) outer side of the balloon includes an adhesion polymer, or (3) a combination of (1) and (2) such that the adhesion polymer exists in an amount effective to cause the stent not to unintentionally dislodge from the balloon.

15. The medical assembly of claim 14, wherein unintentional dislodgement is defined as less than 1 mm in linear and/or radial movement by the stent on the balloon prior to the start of the inflation or expansion of the balloon.

16. The medical assembly of claim 14, wherein unintentional dislodgment is defined as less than 0.5 mm in linear and/or radial movement by the stent on the balloon prior to the start of the inflation or expansion of the balloon.

17. The medical assembly of claim 14, wherein the stent is a bare metallic stent.

18. The medical assembly of claim 14, wherein the stent is a metallic stent having a polymeric drug delivery coating.

19. The medical assembly of claim 14, wherein the stent is a bioabsorbable stent.

20. The medical assembly of claim 14, wherein the stent is a bioabsorbable stent having a polymeric drug delivery coating.

21. The medical assembly of claim 14, wherein the adhesion polymer is selected from the group consisting of poly(ester amide), polyanhydride, polyacid, poly(acrylic acid), poly(methacrylic acid), poly(vinyl alcohol), and combinations thereof.

22. The medical assembly of claim 14, wherein the adhesion polymer is PEA-BZ or PEA-TEMPO.
